(19) United States
(12) Patent Application Publication
(10) Pub. No.: US 2025/0131984 A1
(43) Pub. Date: Apr. 24, 2025

(54) SEQUENCE ERROR CORRECTION USING NEURAL NETWORKS

(71) Applicant: Google LLC, Mountain View, CA (US)

(72) Inventors: Andrew Walker Carroll, Mountain View, CA (US); Gunjan Baid, San Francisco, CA (US); Pi-Chuan Chang, Mountain View, CA (US); Daniel Elwood Cook, Waterville, OH (US); Maria Nattestad, Sunnyvale, CA (US); Taedong Yun, Boston, MA (US); Cory Yuen Fu McLean, Newton, MA (US); MD Kishwar Shafin, Richmond, CA (US); Jean-Philippe Vert, Paris (FR); Quentin Didier Olivier Berthet, Paris (FR); Felipe Llinares López, Paris (FR); Ashish Teku Vaswani, San Francisco, CA (US)

(21) Appl. No.: 18/687,059

(22) PCT Filed: Aug. 29, 2022
(86) PCT No.: PCT/US2022/041920

§ 371 Date: Feb. 27, 2024

Related U.S. Application Data
(60) Provisional application No. 63/238,080, filed on Aug. 27, 2021.

(51) Int. Cl. G16B 30/00; G06N 3/09; G16B 40/20

(52) U.S. Cl. CPC G16B 30/00; G06N 3/09; G16B 40/20

(57) ABSTRACT

Methods, systems, and apparatus, including computer programs encoded on computer storage media, for sequence error correction using neural networks.

### CROSS-REFERENCE TO RELATED APPLICATION

This application claims the benefit of U.S. Provisional Application No. 63/238,080, filed on Aug. 27, 2021. The disclosure of the prior application is considered part of and is incorporated by reference in the disclosure of this application.

### BACKGROUND

This specification relates to processing inputs using neural networks.

Neural networks are machine learning models that employ one or more layers of nonlinear units to predict an output for a received input. Some neural networks include one or more hidden layers in addition to an output layer. The output of each hidden layer is used as input to the next layer in the network, i.e., another hidden layer or the output layer. Each layer of the network generates an output from a received input in accordance with current values of a respective set of parameters.

### SUMMARY

This specification describes a system implemented as computer programs on one or more computers in one or more locations that generates a consensus output sequence from a plurality of initial candidate output sequences using a neural network.

This specification also describes a system implemented as computer programs on one or more computers in one or more locations that trains a sequence generation neural network using an alignment loss.

The subject matter described in this specification can be implemented in particular embodiments so as to realize one or more of the following advantages.

Many systems that perform tasks that require generating output sequences can generate individual output sequences that are error-prone and have a high degree of variability. Some systems attempt to overcome these issues by generating multiple candidate output sequences, e.g., by performing the task multiple different times on the same input, and then selecting one of these candidates as the final output. However, these candidate output sequences will generally contain inconsistencies and determining which is most accurate is therefore difficult. Another complicating factor is that the candidate output sequences may also not be temporally aligned, i.e., the candidates can include “empty” outputs at different positions and can include different numbers of “empty” outputs.

This specification describes a system that uses a neural network, e.g., a Transformer neural network, to correct errors in candidate output sequences, i.e., by resolving inconsistencies and temporal misalignments between multiple different candidate output sequences, to generate a single, consensus output sequence that can be provided as the final output sequence for a given task. By using the described techniques, the consensus output sequence is significantly more accurate than any of the individual output sequences and significantly more accurate than other approaches that attempt to generate consensus output sequences.

As a particular example, the system can be used to perform consensus genomic sequencing, i.e., to generate a consensus genomic “read” sequence from multiple observations of the same molecule, i.e., from multiple individual reads of the same molecule.

In particular, at the read level of genomic sequencing, the higher error of single molecule observations, i.e., of single reads of a given molecule, is mitigated by consensus observations, i.e., by using multiple different reads of the given molecule to generate a single consensus read. For example, in Illumina data, the consensus is spatial, through clusters of amplified molecules. As another example, Pacific Biosciences (PacBio) uses repeated sequencing of a circular molecule to build consensus across time.

The accuracy of these consensus generation approaches, and the manner they fail, ultimately limits the read lengths of these methods and the analyzable regions of the genome. The need to manage sequencing errors through generating consensus sequences constrains the minimum number of passes required for acceptable sequencing accuracy, and therefore the yield and quality of the sequencing process.

The described techniques can significantly improve over conventional consensus generation techniques, e.g., ones that use a Hidden Markov Model (HMM) to generate consensus output sequences, thereby significantly reducing the impact of this constraint and improving the yield and quality of the sequencing process.

More specifically, using the described techniques improves the contiguity, completeness, and correctness of genome assembly when compared to assemblies generated using conventional consensus generation techniques. Further, these improvements in accuracy allow for longer read lengths while retaining acceptable read accuracy, enabling improvements in contiguity of genome assembly and increasing the experimental design options for genomic sequencing.

This specification also describes a technique for training a sequence generation neural network using an alignment loss. Since insertion and deletion (INDEL) errors are the dominant class of errors in training data for genomic sequencing tasks as well as other sequence generation tasks, the alignment loss focuses on penalizing these types of errors and allows the network to be trained to more accurately represent misalignment errors in the training process, resulting in a trained neural network that more accurately generates output sequences than those trained using conventional loss functions.

The details of one or more embodiments of the subject matter of this specification are set forth in the accompanying drawings and the description below.

Other features, aspects, and advantages of the subject matter will become apparent from the description, the drawings, and the claims.

### BRIEF DESCRIPTION OF THE DRAWINGS

FIG. 1 is a diagram of an example error correction system.

FIG. 2 is a flow diagram of an example process for generating a consensus output sequence.

FIG. 3 is a diagram of an example of the operation of the error correction system.

FIG. 4 is a flow diagram of an example process for training a neural network.

Like reference numbers and designations in the various drawings indicate like elements.

### DETAILED DESCRIPTION

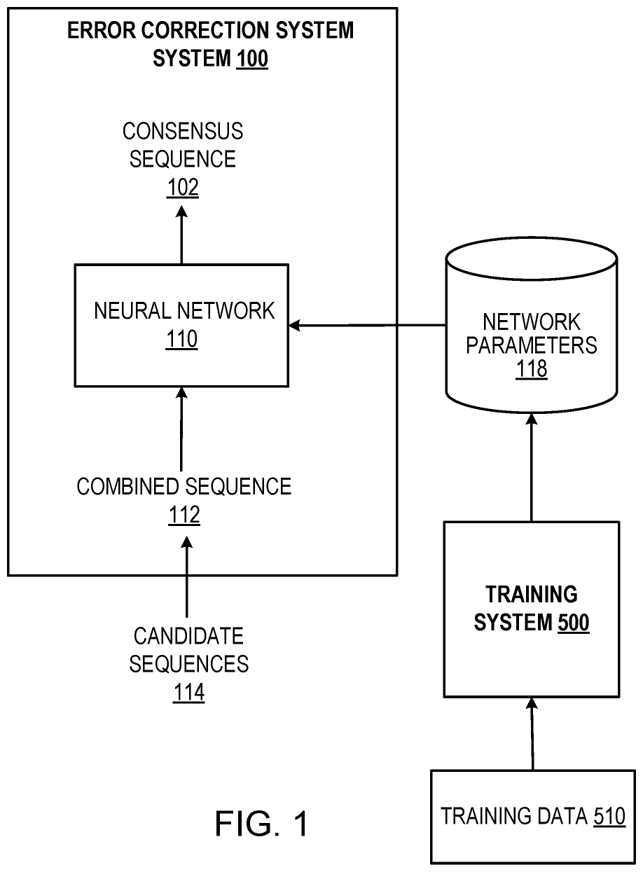

FIG. 1 is a diagram of an example error correction system 100. The error correction system 100 is an example of a system implemented as computer programs on one or more computers in one or more locations, in which the systems, components, and techniques described below can be implemented.

The error correction system 100 is a system that generates a consensus output sequence 150 from a plurality of initial candidate output sequences 102 using a neural network 110.

Generally, the system 100 uses the neural network 110 to analyze an alignment of similar sequences (“candidate output sequences” 102) to generate a consensus output sequence 150 that corrects errors in the similar sequences, e.g., by resolving inconsistencies between the similar sequences.

Generally, the candidate output sequences 102 are generated through some initial analysis performed by another system and can be multiple different “solutions” or “outputs” for an instance of a given task as determined by the other system. That is, the candidate output sequences 102 are generated by another system and are received as input by the system. The other system generates the candidate output sequences 102 by performing an instance of a given task, i.e., by performing the given task multiple times on the same input or starting from the same state. For example, the plurality of candidate output sequences can be a multiple sequence alignment (MSA), e.g., as generated by the system 100 or by the other system, of a plurality of initial candidate output sequences generated by performing the given task multiple times.

Thus, the consensus output sequence 150 generated by the system 100 using the neural network 110 is an error-corrected version of the candidate output sequences 102, i.e., that serves as an output for the same instance of the given task as the candidate output sequences 102.

For example, the candidate output sequences 102 can include genomic sequencing data generated through genomic sequencing, i.e., with the outputs in the candidate output sequences including canonical bases selected from a vocabulary of canonical bases (and, as will be described below, an empty output), and the consensus output sequence 150 is error-corrected genomic sequencing data.

As one example, the similar sequences can be multiple subreads generated through genomic sequencing, e.g., subreads generated by repeatedly sequencing the same molecule or by sequencing the same one or more clusters of amplified molecules. The neural network 110 can then generate a consensus output sequence 150 that corrects sequencing errors in the subreads to generate, as output, an error-corrected, consensus sub-read.

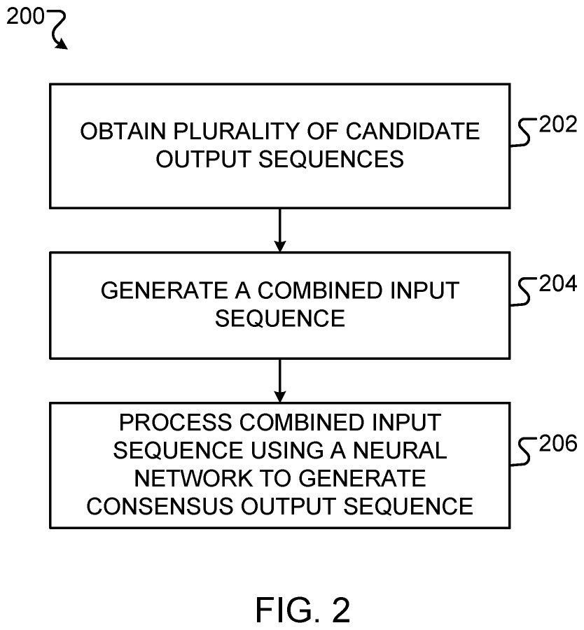

This example is described in more detail below with reference to FIG. 2.

As another example, the candidate output sequences can be Unique Molecular Identifier sequences for a given molecule and the consensus output sequence can be an error-corrected Unique Molecular Identifier sequence for the molecule. Unique molecular identifiers (UMIs) are a type of molecular barcoding that provides error correction and increased accuracy during sequencing. These molecular barcodes are short sequences that can, for example, be used to uniquely tag each molecule in a sample library.

As another example, the candidate output sequences can be Oxford Nanopore Duplex reads, i.e., a sequencing read of a molecule of DNA generated using the Oxford Nanopore “Duplex” technique, and the consensus output sequence can be an error-corrected Oxford Nanopore Duplex read.

As another example, the candidate output sequences can be draft genome assembly sequences, and the consensus output sequence can be an error-corrected genome assembly sequence.

Generally, each candidate output sequence 102 includes a respective output at each of a plurality of positions.

The respective output at any given one of the plurality of positions is either (i) an output from a vocabulary of outputs or (ii) an empty output that is not in the vocabulary. The vocabulary of outputs are the possible outputs for the given task, while the empty output indicates the position should be “blank” or that the initial system could not accurately determine which output from the vocabulary should be included at the position. For example, as will be described in more detail below, the vocabulary of outputs can include a set of canonical bases, while the empty output indicates a “blank” output that is not in the set of bases.

Thus, while all of the candidate output sequences 102 are the same length, i.e., have the same number of positions, they may be inconsistent: i.e., include different numbers of empty outputs, include empty outputs at different positions, or include multiple different vocabulary outputs at the same input position.

To generate the consensus output sequence 150, the system generates a combined input sequence 120 from the candidate output sequences 102.

The combined input sequence 120 includes a respective combined input at each of the plurality of positions, i.e., at each of the positions in the candidate output sequences 102. Generally, the combined input at any given position includes at least, for each of the plurality of candidate output sequences, a respective numeric representation of the respective output at the position in the candidate output sequence.

A “numeric representation” as used in this specification is an ordered collection of numeric values, e.g., a vector of floating point or other numeric values having a pre-determined dimensionality.

As one example, the numeric representation of a given output can be a one-hot encoding of the given output. A one-hot encoding refers to a vector that has a respective dimension corresponding to each output in the vocabulary and to the empty output, with all of the values being 0 except for a value of 1 along the dimension that corresponds to the given output.

As another example, the numeric representation of a given output can be an embedding of the given output. For example, the embedding can be pre-determined or can be learned during the training of the neural network 110.

Thus, the combined input at each position includes information about which output is included at the position in all of the candidate output sequences 102.

As a particular example, the combined input can include a concatenation of the numeric representations of the respective outputs at the position in the plurality of candidate output sequences.

Optionally, as described in more detail below, the combined input can also include additional data in addition to the numeric representations of the respective outputs.

The system 100 then processes the combined input sequence 120 using the neural network 110 to generate the consensus output sequence 150. That is, the system 100 uses the neural network 120 to correct errors and resolve inconsistencies among the candidate output sequences 102 by virtue of processing the combined input sequence 120.

The neural network 110 can have any appropriate architecture that allows the neural network 110 to receive a combined input sequence 120 and to process the combined output 120 to generate the consensus output sequence 150.

As one example, the neural network 110 can be an encoder-only Transformer neural network, i.e., one that has one or more self-attention blocks that repeatedly update the combined input 150 and then generates a score distribution for each position in the output sequence 150 from the output of the last self-attention block.

As another example, the neural network 110 can be an encoder-decoder Transformer neural network that has an encoder neural network that repeatedly applies self-attention to generate an encoded representation of the combined input and then a decoder neural network that auto-regressively generates the output sequence conditioned on the encoded representation, e.g., by alternating between applying cross-attention into the encoded representation and masked self-attention over the currently generated output sequence.

Thus, by effectively representing the candidate output sequences in an input to the neural network 110, the system 100 can leverage the representational capacity of the neural network 110 in order to generate a consensus output sequence 150 that accurately corrects errors in the candidate output sequences 102. That is, because of the structure of the combined input sequence 120, the neural network 110 can effectively process the combined input sequence 120 to generate an accurate output sequence 150. For example, the structure of the combined input sequence 120 can allow the neural network 110 to iteratively update an internal representation of the candidate output sequences by iteratively applying self-attention across the positions in the combined input sequence. By making the predictions of the outputs in the output sequence 150 from these updated internal representations, the neural network 110 can accurately predict how inconsistencies among the candidate output sequences should be resolved.

Prior to using the neural network 110 to generate consensus output sequences, a training system 500 trains the neural network 110 on training data 510, i.e., to determine trained values of the parameters of the neural network 110 from initial values of the parameters. The “parameters” of a neural network refer to the weights and, in some cases, biases of the layers of the neural network. By training the neural network 110 on the training data 510, the training system 500 causes the neural network 110 to accurately generate consensus output sequences.

Generally, the training data 510 includes multiple training examples, with each training example including a network input and a ground truth output sequence that should be generated by processing the network input.

More specifically, each network input includes a set of candidate output sequences and the ground truth output sequence is a ground truth consensus output sequence that should be generated from the candidate output sequences in the network input.

The training system 500 can train the neural network 110 on the training data 510 using any appropriate loss function that accounts for potential differences in alignments between the ground truth output sequence and the predicted output sequence generated by the neural network 110.

For example, the training system 500 can train the neural network 110 to minimize a connection temporal classification (CTC) loss or another sequence transduction loss.

As another example, the training system 500 can train the neural network 110 on an alignment loss that penalizes the neural network 110 for making insertion and deletion (INDEL) errors when generating a predicted consensus output sequence during training.

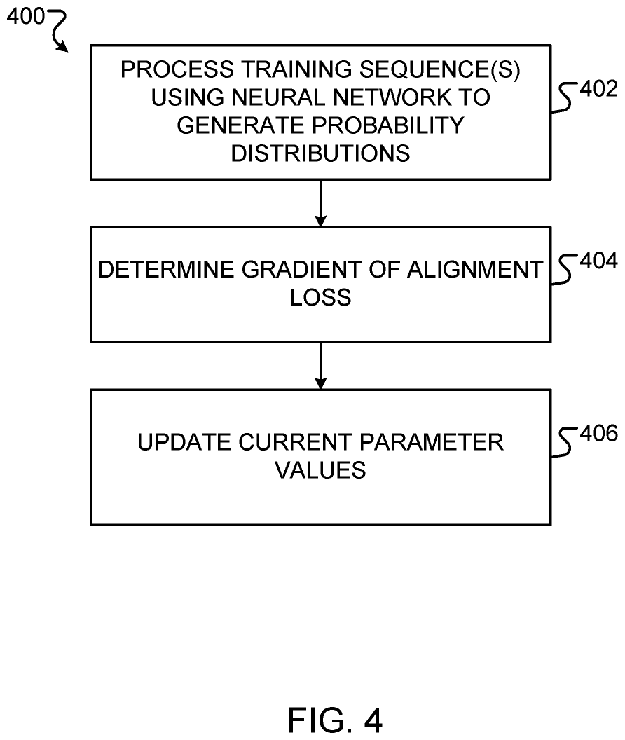

Training the neural network 110 or a different type of sequence generation neural network on this alignment loss is described below with reference to FIG. 4.

FIG. 2 is a flow diagram of an example process 200 for generating a consensus output sequence. For convenience, the process 200 will be described as being performed by a system of one or more computers located in one or more locations. For example, an error correction system, e.g., the error correction system 100 depicted in FIG. 1, appropriately programmed in accordance with this specification, can perform the process 300.

The system obtains a plurality of candidate output sequences (step 202).

Each candidate output sequence includes a respective output at each of a plurality of positions.

As described above, the respective output at each of the plurality of positions is either (i) an output from a vocabulary of outputs or (ii) an empty output that is not in the vocabulary.

An empty output at a given position can indicate that the initial analysis system was uncertain about the actual output at the given position, that the output was not yet complete as of the given position, or can be generated as a result of an error in the processing of the underlying input.

The system generates a combined input sequence from the plurality of candidate output sequences (step 204).

The combined input sequence includes a respective combined input at each of the plurality of positions.

The combined input at any given position, in turn, includes, for each of the plurality of candidate output sequences, a respective numeric representation of the respective output at the position in the candidate output sequence.

Optionally, the combined inputs can also include other data that is relevant to generating the consensus output sequence. In particular, the respective combined input at each of the positions can also include a respective numeric representation of each of one or more auxiliary features for the position.

For example, the respective numeric representation of each of the auxiliary features can be an embedding of the auxiliary feature. In some cases, the embeddings are fixed prior to training the neural network. In other cases, the embeddings are learned while training the neural network, e.g., by the training system 500 of FIG. 1. That is, the embeddings are learned jointly with the training of the neural network.

When the respective combined input at each of the positions also includes a respective numeric representation of each of one or more auxiliary features for the position, the combined input at any given position can be a combination of, e.g., a concatenation of, the respective numeric representations of the respective outputs at the given position in the candidate output sequence and the respective numeric representations of each of one or more auxiliary features for the given position.

As a particular example, the system can have access to an initial predicted consensus output sequence that is generated, e.g., by the initial analysis system that generated the candidate output sequences. For example, the initial predicted consensus output sequence can be generated using a conventional consensus generation technique, e.g., a hidden markov model (HMM)-based technique. In this example, the auxiliary features for each position can include the initial predicted output at the position in the initial predicted consensus output sequence.

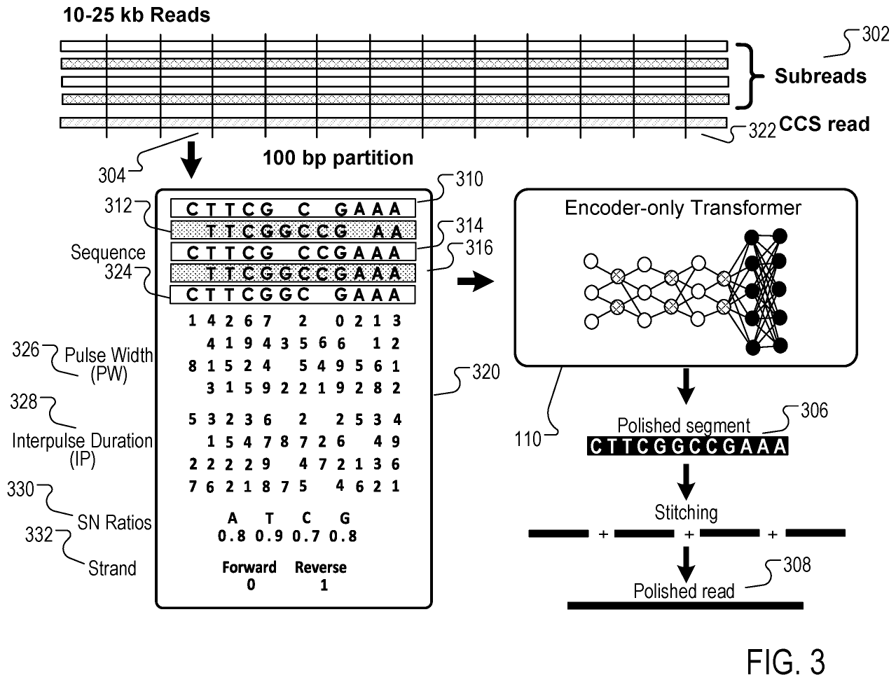

Other examples of auxiliary features will be described below with reference to FIG. 3.

Optionally, at each position, the combined input sequence can also include a positional encoding that identifies the input position, e.g., by using a sine and/or cosine based positional encoding scheme.

The system processes the combined input sequence using a neural network to generate a consensus output sequence that includes a respective output from the vocabulary of outputs at each of a plurality of output positions (step 206).

As a particular example, the neural network can be configured to process the combined input sequence to generate a respective score distribution for each of the plurality of output positions, with each respective score distribution including a respective score for (i) each of the outputs in the vocabulary and (ii) the empty output that is not in the vocabulary.

For example, the neural network can be an encoder-only Transformer or other type of neural network that generates the respective score distributions for each of the output positions in parallel, i.e., in a single forward pass through the neural network. In this example, to account for variable-length prediction, the system can add, to the combined input sequence, a fixed number of padding tokens to the combined input sequence prior to processing the combined input sequence using the neural network. That is, the combined input sequence also includes a predetermined padded combined input at one or more additional positions, e.g., that follow the last position in the candidate output sequences. For example, the padded combined input can be a vector of zeroes or other predetermined values.

As another example, the neural network can be an encoder-decoder Transformer or other type of neural network that generates the respective score distributions auto-regressively, with the score distribution for each position being conditioned on the combined input sequence and the outputs in the consensus output sequence at any earlier positions.

The system can then select the output at each of the output positions using the respective score distribution generated by the neural network for the output position. For example, the system can greedily select the highest scoring output or sample from the score distribution to select the output.

If the selected output for any of the output positions is the empty output, the system can discard, from the consensus output sequence, any output positions for which the selected output is the empty output. That is, the system can remove, from the consensus output sequence all of the empty outputs before finalizing the consensus output sequence.

In some cases, the candidate output sequences correspond to one of a plurality of segments of a larger output sequence. That is, the system receives a respective set of candidates for each of multiple segments of a larger output sequence.

In these implementations, the system can perform the process 200 for each of the segments of the larger output sequence to generate a respective consensus output sequence for each segment. The system can then combine the consensus output sequences for the plurality of segments to generate a larger consensus output sequence, e.g., by concatenating the consensus output sequences for the plurality of segments.

FIG. 3 shows an example of the operation of the system 100. In particular, in the example of FIG. 3, the candidate output sequences are subreads 302 of a genomic sequencing read generated through genomic sequencing.

For example, the subreads 302 can be subreads of the same molecule or of the same clusters of one or more amplified molecules, with each subread being taken from a different spatial location or angle, taken at a different time, or both.

Thus, in the example of FIG. 3, the outputs in the vocabulary are canonical bases: adenine (A), cytosine (C), guanine (G), and thymine (T). The outputs in the candidate output sequences also include an “empty” output.

The output sequence in the example of FIG. 3 is an error-corrected subread that includes only canonical bases and no empty outputs.

More specifically, as shown in FIG. 3, a larger read is divided into multiple segments (also referred to as “partitions”) 304. In the example of FIG. 3, the segments are 100 base pair (bp) partitions.

The system 100 processes the candidate outputs corresponding to each segment to generate a consensus segment 306 (also referred to as a “polished segment”) and then stitches (combines) the polished segments 306 together to generate a consensus read (“polished read”) 308.

As shown in the example of FIG. 3, the system combines the polished segments by concatenating the polished segments one after the other.

FIG. 3 shows a simplified example of the generation of a single consensus segment from four candidate output segments 310, 312, 314, 316.

In particular, the first candidate output segment 310 is the sequence [C, T, T, C, G, empty, C, empty, G, A, A, A], the second candidate output segment 312 is the sequence [empty, T, T, C, G, G, C, C, G, empty, A, A], the third candidate output segment 314 is the sequence [C, T, T, C, G, empty, C, C, G, A, A, A], and the fourth candidate output sequence 316 is the sequence [empty, T, T, C, G, G, C, C, G, A, A, A].

Thus, while for some positions the four candidate output sequences have the same output, for other positions different ones of the four candidate output segments have different outputs.

The system 100 generates the polished segment by generating a combined input sequence 320 and processing the combined input sequence using the neural network 110. As shown in FIG. 3, the neural network 110 is an encoder-only Transformer that repeatedly applies self-attention over the combined input sequence 320. However, as described above, the neural network 110 can have any appropriate architecture.

The combined input sequence 320 includes data representing a variety of different information about the candidate output sequences.

In particular, as can be seen from FIG. 3, the combined input sequence 320 includes twelve positions, one corresponding to each position in the candidate output sequences.

At each position, the combined input sequence 320 includes a numeric representation of the output at that position in each of the four combined output sequences. For example, at the first position, the combined input sequence includes numeric representations of, e.g., embeddings of, the base “C”, the empty output, the base “C”, and the empty output.

The combined input sequence 320 also includes, at each position, numeric representations of, e.g., embeddings of, each of a set of auxiliary features.

As described above, in the example of FIG. 3, the system has access to an initial consensus sequence 324 corresponding to the segment (i.e., a corresponding segment 324 of a “CCS read” 322 that is made up of the sequence [C, T, T, C, G, G, C, empty, G, A, A, A].

Thus, combined input sequence includes, at each position, a numeric representation of the corresponding output in the initial consensus sequence.

In the example of FIG. 3, the auxiliary features also include, for each candidate output sequence, a pulse width 326 for the candidate output sequence that includes a respective pulse width value for each non-empty position in the candidate output sequence, e.g., as measured by a basecaller during generation of the candidate sequence.

In the example of FIG. 3, the auxiliary features also include, an interpulse duration 328 that includes a respective interpulse duration value for each non-empty position in the candidate output sequence, e.g., as measured by a basecaller during generation of the candidate sequence.

In the example of FIG. 3, the auxiliary features also include a signal to noise ratio (SN) 330 for the sequencing reaction and the strand 332 of each subread. When, like the SN ratio and the strand features, the features do not include a respective value for each position, the system can repeat the numeric representation for the feature across all of the positions in the combined input sequence.

Once the combined input sequence representing the segment is generated, the system processes the combined input sequence representing the segment using the neural network to generate the polished segment 306, i.e., the consensus output sequence for the segment. In the example of FIG. 3, the polished segment is [C, T, T, C, G, G, C, C, G, A, A, A]. As can be seen in the example of FIG. 3, the polished segment improves upon the initial consensus output sequence due to the structure of the combined input sequence and the representational power of the neural network 110.

FIG. 4 is a flow diagram of an example process 400 for training a neural network using an alignment loss. For convenience, the process 400 will be described as being performed by a system of one or more computers located in one or more locations. For example, a training system, e.g., the training system 500 depicted in FIG. 1, appropriately programmed in accordance with this specification, can perform the process 400.

The system can repeatedly perform iterations of the process 400 on different batches of training examples to train the neural network, i.e., to repeatedly adjust the values of the parameters of the neural network. That is, at each iteration of the process 400, the system obtains a batch of one or more training examples, e.g., by sampling the batch from a larger set of training data, and then performs an iteration of the process 400 to update the current values of the network parameters as of the iteration.

For example, the system can continue to perform iterations of the process 400 until a termination criterion has been satisfied, e.g., until a threshold number of training iterations have been performed, until a specified amount of time has elapsed, or until the parameters have been determined to converge.

As described above, the process 400 can be used to train the neural network 110, i.e., a neural network that generates a consensus output sequence from a combined input sequence. More generally, however, the process 400 can be used to train any of a variety of sequence generation neural networks, i.e., neural networks that generate output sequences conditioned on some network input.

Generally, the process 400 and the alignment loss described below can be used to train any of a variety of neural networks that generate output sequences that may have a different number of outputs than the corresponding ground truth output sequence would have, e.g., if the output vocabulary includes an “empty” token. That is, during training on a given training example, the neural network can generate an output sequence with a different number of outputs than the number of outputs in the ground truth output for the training example, i.e., the output that should be generated by the neural network for the training example.

In particular, in addition to training neural networks that generate consensus output sequences like those described above, the alignment loss can be applied to train neural networks to perform any of a large number of other machine learning tasks, e.g., machine translation, speech recognition, language modeling, and so on.

A more detailed description of possible tasks that can be performed by the sequence generation neural network that is trained using the process 400 is provided below.

As described above, to perform an iteration of the process 400, the system obtains a batch of one or more training examples. Each training example has a network input and a corresponding ground truth output sequence that should be generated by the neural network by processing the network input. The ground truth output sequence has a respective ground truth output at each of a plurality of ground truth positions and each respective ground truth output is selected from a vocabulary of outputs, i.e., the ground truth output sequence does not include any empty outputs.

For each training example in the batch, the system processes the training network input in the training example using the neural network and in accordance with current values of the network parameters to generate a respective probability distribution for each of a plurality of output positions in a training output sequence (step 402). The respective probability distribution for each of the plurality of output positions includes a respective probability for (i) each output in the vocabulary and (ii) an empty output.

As described above, the number of output positions in the training output sequence can be different from the number of ground truth output positions in the ground truth output sequence. For example, the neural network can be configured to generate respective probability distributions for each of a fixed number of output positions that is generally greater than the total number of positions in any one ground truth output sequence. After training, because empty outputs are discarded, output sequences of the correct length can be accurately generated based on probability distributions output by the neural network.

The system determines, for each training example, a respective gradient with respect to the parameters of a loss function (an “alignment loss”) (step 404).

The loss function measures a respective loss for each of a set of one or more alignments between the output positions and the ground truth positions in the ground truth output sequence.

An “alignment” assigns each output position in a respective subset of the output positions (in the training output sequence) to a corresponding ground truth position in a respective subset of the ground truth positions in the ground truth output sequence in the training example. If an alignment assigns a first output position to a first ground truth position, the output at the first output position in a given sequence generated by the neural network is a prediction of the first ground truth position in the corresponding ground truth output sequence.

More specifically, an alignment π of length k is an increasing subset of k positions between output positions y and ground truth output positions t, where

π= {1<π (y, 1)<π (y, 2)< . . . <π (y, k)<, 1<π (t, 1)<π (t, 2)< . . . <π (t, k)<M} in both y and t, where N is the total number of output positions, M is the total number of ground truth positions, such that position x (y, v) in y predicts position n (t, v) in t, for v=1 . . . , k.

Generally, the set of alignments can include any number of alignments. For example, the set of alignments can include each possible alignment for each value of k up to a predetermined maximum value. As yet another example, the set of alignments can include each possible alignment between a predetermined minimum value of k and the predetermined maximum value.

The respective loss for each of the one or more alignments measures at least, (i) for each output position that is not assigned to any ground truth position by the alignment, a cross-entropy loss between the respective probability distribution for the output position and a target probability distribution that assigns a probability of one to the empty output and (ii) for each output position that is assigned to a corresponding ground truth position by the alignment, a cross-entropy loss between the respective probability distribution for the output position and a target probability distribution that assigns a probability of one to the ground truth output at the corresponding ground truth position that is assigned to the output position by the alignment.

Optionally, the respective loss for each of the alignments can also include, for each ground truth position that is not assigned to any output positions by the alignment, a constant loss value.

Generally, the respective loss for each alignment can be a sum or a weighted sum of (i) for each output position that is not assigned to any ground truth position by the alignment, the cross-entropy loss between the respective probability distribution for the output position and the target probability distribution that assigns a probability of one to the empty output, (ii) for each output position that is assigned to a corresponding ground truth position by the alignment, the cross-entropy loss between the respective probability distribution for the output position and the target probability distribution that assigns a probability of one to the ground truth output at the corresponding ground truth position that is assigned to the output position by the alignment, and, optionally, (iii) for each ground truth position that is not assigned to any output positions by the alignment, the constant loss value.

As a specific example, the loss loss_{π}(y, t) for an alignment π can satisfy:

$${loss}_{π} ( y , t ) = {\sum }_{v = 1}^{k} {loss}_{CE} ( {y}_{π ( y , v )} , {t}_{π ( t , v )} ) + {\sum }_{v ∈ π ( \overline{y} )} {loss}_{CE} ( {y}_{v} , \$ ) + {\sum }_{v ∈ π ( \overline{t} )} γ ,$$

where loss_{CE} refers to a cross-entropy loss, t_{π(t,v)} is the target probability distribution that assigns a probability of one to the ground truth output at the corresponding ground truth position π(t, v) that is assigned to the output position π(y, v) by the alignment, π(y̅) is the set of output positions not assigned to any ground truth position by the alignment, $ represents the target probability distribution that assigns a probability of one to the empty output, π(t̅) is the set of ground truth positions not assigned to any output position in the alignment, and γ is a fixed error value that is greater than zero (or equal to zero when (iii) is not included in the loss).

By framing the loss as described above, the loss penalizes the neural network for making insertion and deletion errors when generating a given prediction.

The overall value of the loss function is determined based on the respective losses for the set of alignments.

In one example, the overall value of the loss can be the minimum of the respective losses for the one or more alignments.

In another example, the overall value of the loss can be the smooth minimum of the respective losses of the one or more alignments. In this example, the overall value of the loss can satisfy:

$$- ϵlog ( {\sum }_{π} {e}^{- {loss}_{π} ( y , t ) / ϵ} ,$$

where the sum is over the set of alignments π and ε≥0 is a parameter that controls how suboptimal alignments contribute to the loss. At the limit ε=0, the system computes the loss based only on the best alignment loss, i.e., the smooth minimum becomes the minimum, while setting ε>0 allows the system to create a smoother loss function by using a smooth minimum.

Because the set of alignments can include a large number of alignments, the system can evaluate the loss function and determine the respective gradients through differentiable dynamic programming, i.e., to ensure that the training remains computationally efficient. Computing gradients through differentiable dynamic programming is described in more detail in Mensch, A. & Blondel, M. Differentiable Dynamic Programming for Structured Prediction and Attention. 80, 3462-3471 (2018).

The system then updates the current values of the parameters of the neural network using the respective gradients for the training examples in the batch (step 406). For example, the system can combine, e.g., sum or average, the respective gradients for the training examples, and then apply an optimizer, e.g., Adam, rmsprop, or another appropriate optimizer, to the combined gradient and the current values of the parameters to generate updated values of the network parameters.

Some example sequence generation tasks for which the alignment loss can be used follow.

As one example, the task may be a machine translation task. That is, if the input to the neural network is a sequence of text in one language, the output generated by the neural network may be a piece of text in another language that is predicted to be a proper translation of the input text into the other language.

As another example, the task may be an audio processing task. An example of such a task is speech recognition, where, if the input to the neural network is a sequence representing a spoken utterance, the output generated by the neural network is a piece of text that is predicted to be the correct transcript for the utterance.

As another example, the task can be a text to speech task, where the input is text in a natural language or features of text in a natural language and the network output is a spectrogram, a waveform, or other data defining audio of the text being spoken in the natural language.

As another example, the task can be a text generation task, where the input is a sequence of text, and the output is another sequence of text, e.g., a completion of the input sequence of text, a response to a question posed in the input sequence, or a sequence of text that is about a topic specified by the first sequence of text. As another example, the input to the text generation task can be an input other than text, e.g., an image, and the output sequence can be text that describes the input.

As another example, the task can be an image generation task, where the input is a conditioning input and the output is a sequence of intensity value inputs for the pixels of an image.

As another example, the task can be an agent control task, where the input is a sequence of observations or other data characterizing states of an environment and the output defines an action to be performed by the agent in response to the most recent data in the sequence. The agent can be, e.g., a real-world or simulated robot, a control system for an industrial facility, or a control system that controls a different kind of agent.

As another example, the task can be a genomics task, where the input is a sequence representing a fragment of a DNA sequence or other molecule sequence and the output is either an embedding of the fragment for use in a downstream task, e.g., by making use of an unsupervised learning technique on a data set of DNA sequence fragments, or an output for the downstream task. Examples of downstream tasks include promoter site prediction, methylation analysis, predicting functional effects of non-coding variants, and so on.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, e.g., one or more modules of computer program instructions encoded on a tangible non transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a data communication network.

In this specification, the term “database” is used broadly to refer to any collection of data: the data does not need to be structured in any particular way, or structured at all, and it can be stored on storage devices in one or more locations. Thus, for example, the index database can include multiple collections of data, each of which may be organized and accessed differently.

Similarly, in this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine: in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non volatile memory, media and memory devices, including by way of example semiconductor memory devices, e.g., EPROM, EEPROM, and flash memory devices: magnetic disks, e.g., internal hard disks or removable disks: magneto optical disks; and CD ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well: for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user: for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, e.g., inference, workloads.

Machine learning models can be implemented and deployed using a machine learning framework, e.g., a TensorFlow framework.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. A method performed by one or more computers, the method comprising:
obtaining a plurality of candidate output sequences, each candidate output sequence comprising a respective output at each of a plurality of positions, wherein the respective output at each of the plurality of positions is either (i) an output from a vocabulary of outputs or (ii) an empty output that is not in the vocabulary;
generating a combined input sequence from the plurality of candidate output sequences, the combined input sequence comprising a respective combined input at each of the plurality of positions, wherein the respective combined input at each of the plurality of positions comprises:
for each of the plurality of candidate output sequences, a respective numeric representation of the respective output at the position in the candidate output sequence; and
processing the combined input sequence using a neural network to generate a consensus output sequence that includes a respective output from the vocabulary of outputs at each of a plurality of output positions.

2. The method of claim 1, wherein the neural network is configured to process the combined input sequence to generate a respective score distribution for each of the plurality of output positions, and wherein each respective score distribution comprises a respective score for (i) each of the outputs of the vocabulary and (ii) the empty output that is not in the vocabulary.

3. The method of claim 2, wherein processing the combined input sequence using a neural network to generate a consensus output sequence that includes a respective output from the vocabulary of outputs at each of a plurality of output positions comprises:
selecting an output at each of the output positions using the respective score distribution generated by the neural network for the output position.

4. The method of claim 3, wherein processing the combined input sequence using a neural network to generate a consensus output sequence that includes a respective output from the vocabulary of outputs at each of a plurality of output positions further comprises:
discarding, from the consensus output sequence, any output positions for which the selected output is the empty output.

5. The method of claim 2, wherein each output position corresponds to a respective position in the combined input sequence, and wherein the neural network is a self-attention neural network that comprises:
a sequence of one or more self-attention blocks that each update the respective combined input at each position in the combined input sequence; and
one or more output layers that, for each position in the combined input sequence, process the updated combined input for the position generated by the last self-attention block in the sequence to generate the score distribution for the position.

6. The method of claim 5, wherein the combined input sequence further comprises a predetermined padded combined input at one or more additional positions.

7. The method of claim 1, wherein the respective numeric representation of the respective output at the position in the candidate output sequence is an embedding of the respective output.

8. The method of claim 7, wherein the embeddings of the outputs in the vocabulary and the empty output are learned while training of the neural network.

9. The method of claim 1, wherein the respective combined input at each of the plurality of positions comprises a concatenation of the respective numeric representations of the respective outputs at the position in the plurality of candidate output sequences.

10. The method of claim 1, wherein the plurality of candidate output sequences are a multiple sequence alignment (MSA) of a plurality of initial candidate output sequences.

11. The method of claim 1, wherein the respective combined input at each of the plurality of positions further comprises a respective numeric representation of each of one or more auxiliary features for the position.

12. The method of claim 11, wherein the respective numeric representations of each of the auxiliary features are embeddings of the auxiliary features.

13. The method of claim 12, wherein the embeddings of the auxiliary features are learned while training the neural network.

14. The method of claim 11, wherein the respective combined input at each of the plurality of positions comprises a concatenation of the respective numeric representations of (i) the respective outputs at the position in the plurality of candidate output sequences and (ii) the one or more auxiliary features for the position according to a predetermined order.

15. The method of claim 1, wherein the candidate output sequences comprise genomic sequencing data generated through genomic sequencing, wherein the outputs in the vocabulary are canonical bases, and wherein the consensus output sequence is error-corrected genomic sequencing data.

16. The method of claim 1, wherein the candidate output sequences are subreads of a genomic sequencing read generated through genomic sequencing, wherein the outputs in the vocabulary are canonical bases, and wherein the consensus output sequence is an error-corrected subread.

17. The method of claim 16, wherein the respective combined input at each of the plurality of positions further comprises a respective numeric representation of each of one or more auxiliary features for the position, and wherein the auxiliary features include one or more of:
an initial consensus subread sequence;
a pulse width;
an interpulse duration;
a signal-to-noise ratio; or
strand information.

18. The method of claim 1, wherein the candidate output sequences are Unique Molecular Identifier sequences, and wherein the consensus output sequence is an error-corrected Unique Molecular Identifier sequence.

19. The method of claim 1, wherein the candidate output sequences are Oxford Nanopore Duplex reads, and wherein the consensus output sequence is an error-corrected Oxford Nanopore Duplex read.

20. The method of claim 1, wherein the candidate output sequences are draft genome assembly sequences, and wherein the consensus output sequence is an error-corrected genome assembly sequence.

21. The method of claim 1, wherein the candidate output sequences correspond to one of a plurality of segments of a larger output sequence, and wherein the method further comprises, for each other segment of the larger output sequence:
obtaining a plurality of candidate output sequences corresponding to the other segment, each candidate output sequence comprising a respective output from the vocabulary of outputs at each of a plurality of positions;
generating a combined input sequence corresponding to the other segment from the plurality of candidate output sequences corresponding to the other segment, the combined input sequence comprising, at each of the plurality of positions:
for each of the plurality of candidate output sequences corresponding to the other segment, a respective numeric representation of the respective output at the position in the candidate output sequence corresponding to the other segment;
processing the combined input sequence using the neural network to generate a consensus output sequence corresponding to the other segment that includes a respective output from the vocabulary of outputs at each of a plurality of output positions; and
combining the consensus output sequences for the plurality of segments to generate a larger consensus output sequence.

22. A method of training a neural network having a plurality of parameters and configured to process a network input to generate an output sequence from the network input, the method comprising:
obtaining a batch of one or more training examples, each training example comprising (i) training network input and (ii) a ground truth output sequence that comprises a respective ground truth output at each of a plurality of ground truth positions, wherein each respective ground truth output is selected from a vocabulary of outputs;
for each training example in the batch:
processing the training network input in the training example using the neural network and in accordance with current values of the network parameters to generate a respective probability distribution for each of a plurality of output positions in a training output sequence, wherein the respective probability distribution for each of the plurality of output positions comprises a respective probability for (i) each output in the vocabulary and (ii) an empty output;
determining, for the training example, a respective gradient with respect to the parameters of a loss function that measures a respective loss for each of one or more alignments between the output positions and the ground truth positions in the ground truth output sequence,
wherein each alignment assigns each output position in a respective subset of the output positions to a corresponding ground truth position in a respective subset of the ground truth positions in the ground truth output sequence in the training example, and
wherein the respective loss for each of the one or more alignments measures at least:
(i) for each output position that is not assigned to any ground truth position by the alignment, a cross-entropy loss between the respective probability distribution for the output position and a target probability distribution that assigns a probability of one to the empty output; and
(ii) for each output position that is assigned to a corresponding ground truth position by the alignment, a cross-entropy loss between the respective probability distribution for the output position and a target probability distribution that assigns a probability of one to the ground truth output at the corresponding ground truth position that is assigned to the output position by the alignment; and
updating the current values of the parameters of the neural network using the respective gradients for the training examples in the batch.

23. The method of claim 22, wherein the respective loss for each of the one or more alignments also measures:
(iii) for each ground truth position that is not assigned to any output positions by the alignment, a constant loss value.

24. The method of claim 23, wherein the respective loss for each of the alignments is a sum or a weighted sum of:
(i) for each output position that is not assigned to any ground truth position by the alignment, the cross-entropy loss between the respective probability distribution for the output position and the target probability distribution that assigns a probability of one to the empty output;
(ii) for each output position that is assigned to a corresponding ground truth position by the alignment, the cross-entropy loss between the respective probability distribution for the output position and the target probability distribution that assigns a probability of one to the ground truth output at the corresponding ground truth position that is assigned to the output position by the alignment; and
(iii) for each ground truth position that is not assigned to any output positions by the alignment, the constant loss value.

25. The method of claim 22, wherein the loss function measures a smooth minimum over the respective losses for the one or more alignments.

26. The method of claim 22, wherein the loss function measures a minimum over the respective losses for the one or more alignments.

27. The method of claim 22, wherein determining, for the training example, the respective gradient of the loss function comprises determining the respective gradient through differentiable dynamic programming.

28. The method of claim 22, wherein a number of ground truth positions in the ground truth output sequence is different from a number of output positions in the training output sequence.

29. (canceled)

30. The method of claim 22, wherein the network input comprises a genomic sequencing data generated through genomic sequencing and the output sequence generated from the network input comprises error-corrected genomic sequencing data.

31. (canceled)

32. A system comprising:
one or more computers; and
one or more storage devices storing instructions that, when executed by the one or more computers, cause the one or more computers to perform operations comprising:
obtaining a plurality of candidate output sequences, each candidate output sequence comprising a respective output at each of a plurality of positions, wherein the respective output at each of the plurality of positions is either (i) an output from a vocabulary of outputs or (ii) an empty output that is not in the vocabulary;
generating a combined input sequence from the plurality of candidate output sequences, the combined input sequence comprising a respective combined input at each of the plurality of positions, wherein the respective combined input at each of the plurality of positions comprises:
for each of the plurality of candidate output sequences, a respective numeric representation of the respective output at the position in the candidate output sequence; and
processing the combined input sequence using a neural network to generate a consensus output sequence that includes a respective output from the vocabulary of outputs at each of a plurality of output positions.

33. (canceled)
